(19) United States
(12) Patent Application Publication
(10) Pub. No.: US 2011/0070340 A1
(43) Pub. Date: Mar. 24, 2011

(54) Method for Controlling at Least Two Cooking Devices, Cooking Device, and System Including at Least Two Cooking Devices

(75) Inventors: Stefan Pechaigner, Dachau (DE); Gerhard Kramer, Penzig/Untermuhlhausen (DE); Markus Lingenheil, Breisach/Oberrimsingen (DE); Regina Hoemme, Augsburg (DE); Manfred Breunig, Schongau (DE)

(73) Assignee: RATIONAL AG, Landsberg/Lech (DE)

(21) Appl. No.: 12/866,767

(22) PCT Filed: Jan. 22, 2009
(86) PCT No.: PCT/DE09/00082

§ 371 Date: Dec. 8, 2010

(30) Foreign Application Priority Data
Feb. 18, 2008 (DE) 10 2008 009 659.8

(51) Int. Cl. A47J 36/00; G01N 33/02

(52) U.S. Cl. 426/231; 99/331

(57) ABSTRACT

The invention relates to a method for controlling at least two cooking devices, an associated cooking device and an associated system consisting of at least two cooking devices. According to the invention, data is generated in a control unit of at least one cooking device, said data being suitable for influencing the operation of another cooking device. The data is transmitted to a control unit (24) of the other cooking device and the control unit of the other cooking device controls the operation of said other cooking device (24) in accordance with the transmitted data.

### CROSS-REFERENCE TO RELATED APPLICATIONS

This is the U.S. National Stage of PCT/DE2009/000082, filed Jan. 22, 2009, and which claims the benefit of priority of DE 10 2008 009 659.8, filed Feb. 18, 2008, the entire contents of which are hereby incorporated herein by reference.

### FIELD OF THE DISCLOSURE

The present disclosure concerns a method for controlling at least two cooking devices, a cooking device which is suitable for such a method, as well as a system which comprises at least two such cooking devices.

### BACKGROUND

Modern cooking devices are designed to cook foods in many different ways, for example by baking, roasting, grilling, steaming, stewing, blanching, poaching, etc. All these types of food preparation are referred to in the present document under the collective term “cooking.” Furthermore, modern cooking appliances allow the preparation of different foods simultaneously in the same cooking device corresponding to their optimum cooking requirements, without any transfer of flavor occurring. Thus, a single cooking device can roughly speaking overtake the function of a complete conventional kitchen. Although in some kitchens, in particular in the gastronomical area, several cooking devices are used simultaneously, due to the versatility of each individual cooking device, these are normally operated completely independently of one another.

DE 202 03 117 U1 shows a control of operation for different oven modules. Hereby, a multiple number of oven modules can be operated through a single operating unit, which is connected with the oven modules through a USB connection. This control of operation thus allows a remote control of one or several oven modules through an additional central operating unit.

### GENERAL DESCRIPTION OF THE DISCLOSURE

The object of the present disclosure is to provide a method for controlling two or more cooking devices which allows an optimal use of a multiple number of cooking devices. A further object of the present disclosure is to provide a corresponding cooking device and a system comprising of corresponding cooking devices.

A cooking device according to the present disclosure comprises a control unit which is suitable for producing data which are suitable for influencing the operation of a further cooking device and for receiving data from a further cooking device and for controlling the operation of its own cooking device in dependence on these data.

With the possibility of a cooking device for producing data which are suitable for influencing the operation of a further cooking device, or the possibility of receiving such data from a further cooking device, the cooking operation in different cooking devices can be synchronized. The disclosure is based on the surprising finding that although each cooking device individually is self-sufficient and is able to almost any conceivable type of cooking operation, additional advantages are provided if these self-sufficient devices are synchronized to one another. Hereby the term “synchronization” is to be understood in a broad sense and it should include any type of adjustment of cooking operations of different cooking devices to one another. A number of useful examples of such a synchronization is described below.

Preferably, the control unit is suitable for transferring data to the further cooking device without the interconnection of a further data processing unit. Thus, for the purpose of synchronization of the cooking operations, the cooking devices can be connected to one another communicatively without another data processing unit, for example without a server or another external control unit, having to be connected in-between. This facilitates the installation of a system consisting of several such cooking devices, the subsequent addition of additional cooking devices and the operation, because no additional external data processing unit has to be operated. Hereby the control units of the different cooking devices preferably have equal rights, that is, each device can equally produce data for another cooking device and each cooking device can equally receive such data and control its operation in dependence on the received data. In computer technology, such a networking of control units would be called peer-to-peer connection. This type of connection has the advantage that the cooking devices, which are self-sufficient and have equal rights, can simply be connected to one another without any additional network administration activity.

Preferably the control unit is suitable for sending the data in a broadcast to the control units of all cooking devices with which it is communicatively connected. Furthermore, the control unit is suitable for receiving a broadcast that was sent by the control unit of a further cooking device, to extract data from the broadcast which are relevant for the operation of its own cooking device, and to control the corresponding cooking device in accordance with the extracted data.

Such a broadcast is also referred to as “broadcasting” in computer technology. Hereby data can be sent to the control units of all cooking devices which are connected to one another according to a so-called “broadcasting protocol”. Hereby the individual control units are programmed in such a way that they can extract from the data contained in the broadcast the ones which are relevant for the operation of its own cooking device and then control its own cooking device according to these extracted data.

Preferably the control unit for producing data for a further cooking device is identical with the control unit used for controlling the cooking operation of its own cooking device, that is, the one belonging to the control unit. In this embodiment, the production of data for further cooking devices represents an additional function added to the control of its own cooking device, which can be realized, for example, by the same microcontroller. As always, in this embodiment the individual cooking devices are self-sufficient, but can adjust their operations to one another by data exchange. Thereby differs this embodiment of the disclosure , for example, from a remote control of a device in which the data relevant for the control are produced, for example, on a remote computer and are sent to a device through the internet.

The data that are produced in the cooking device for use by a further cooking device can be fundamentally of any arbitrary type as long as they are suitable for influencing the operation of the further cooking device. In particular these data represent information regarding the device's own current operation which have to be taken into consideration in the course of the desired synchronization with a further cooking device. This information may include, for example, the signaling of errors in its own device. When this error signal is transferred to a further cooking device, said further cooking device can adjust its operation correspondingly. For example, during the error signaling it may stop or slow down its own cooking process so that foods which are prepared in both cooking devices can be cooked to their end at the same time in spite of the error.

Other examples of information which can be represented by the said data are the current climate parameters in the cooking chamber of the data-producing cooking device and/or the remaining cooking time in the cooking device which produces the data. Then these data can influence the cooking operation in the further cooking device, for example by adjusting its cooking operation to the remaining cooking time or by adjusting the climate conditions in the cooking chamber. This exchange of information can occur mutually so that the cooking devices can adjust their cooking operations to one another.

Another example of information regarding the cooking operation in the data-producing device can be information regarding resources which are used by the particular cooking device. When certain resources are limited, the data-receiving cooking devices can determine how much of the limited resources are still available, and then adjust their cooking operation to that. An example for such a limited resource is the maximum frying output provided in the kitchen.

For example, above a certain frying output it is prescribed that a stationary fire extinguisher be installed. When several cooking devices are installed in a kitchen, each of which provides a frying function as one of the large number of cooking functions, such a maximum allowable frying output is reached rapidly even though in practice there is practically never any necessity to fry in all devices simultaneously, so that the theoretically-possible frying output is never reached in practice. Then each cooking device can produce data which represent what output the frying proceeds in it, and these data are sent to all other cooking devices. When, in another cooking device frying should be performed, the particular cooking device determines, based on the data which were sent to it from the further cooking devices, what frying output is being used overall presently in the further cooking devices. This present total frying output of the further cooking devices is deducted from the maximum permissible flying output. When the difference thus determined is sufficient for its own flying operation, then this is begun. Otherwise it is denied. In this way it is ensured that the permissible maximum permissible frying output (without stationary fire extinguisher devices) is never exceeded in the kitchen.

The exclusion of simultaneous frying with more than a predetermined total frying output is an example of the above-mentioned synchronization of the cooking devices.

However, the data produced in a control unit which are suitable for influencing the operation of a further cooking device may also represent commands. Examples of such commands are cooking commands, which initiate certain cooking functions in the data-receiving cooking device, or locking commands, which lock certain functions in the data-receiving cooking device. An example of a locking command could again concern a frying function. For example, in a simple embodiment it could be determined that only one cooking device may fry at a given time. In this case the control unit of the cooking device in which the frying should be performed sends a locking command to the further cooking devices which prevents frying in the further cooking devices.

In an advantageous embodiment the data may be suitable for influencing the operation in two cooking devices in such a way that the cooking conditions in the two cooking devices correspond to one another. For example, when large amounts of a food are to be prepared for which there is no room in a single cooking device, the foods can simply be divided between two cooking devices. By means of the exchanged data it is ensured that the cooking operation that is being carried out in a signaling cooking device is carried out identically in the further cooking device, so that the foods are prepared at the same time in the two cooking devices and in an identical manner. Therefore the operator must monitor the cooking operation only in one of the cooking devices and then rely on the fact that the other foods in the further cooking device are being cooked in an identical manner. Effectively, as a result of this function, two essentially self-sufficient and independent cooking devices can be used to create a cooking device with twice the capacity. This function represents another example of the above-mentioned synchronization of cooking devices.

In an advantageous embodiment, the control unit is suitable for controlling its own cooking device after receiving time information from a further cooking device in such a way that the cooking process is finished in its own cooking device in accordance with the time information. In this way, different cooking processes in different cooking devices can be synchronized in such a way that they are finished simultaneously or at least approximately simultaneously, as a result of which different foods can be cooked to completion exactly simultaneously for serving. When in one of the participating cooking devices an unforeseen disturbance occurs, for example, the door of the cooking device was opened or a brief lack of water occurred, all further cooking devices can be informed of that by a broadcast. The further participating cooking devices can then adjust their cooking time in such a way that in spite of the temporary disturbance all cooking processes are ended at the same time.

Preferably the cooking device comprises an input device for entering cooking commands for the corresponding cooking device and for entering information concerning the synchronization of the operation of the corresponding cooking devices with at least one further cooking device. According to this embodiment, thus the same input device which is provided for operating an individual cooking device can also be used for the input of information that concerns the synchronization of two or more cooking devices. For the operator this has the advantage that the operation is fundamentally the same as that which the operator already knows from completely separate cooking devices, only with the additional function of adjusting the cooking operation of the separate cooking devices to one another, for example by prescribing that the cooking processes in the two cooking devices should end at the same time.

Preferably the cooking device also comprises a display for displaying information concerning the operation of the corresponding cooking device, whereby the control unit of the corresponding cooking device is additionally suitable for displaying information concerning the operation of a further cooking device by its own display device. Similarly as in the case of the input device, the display thus has here fundamentally the conventional function that the operator is already familiar with, to display information regarding the operation of its own cooking device. However, additionally the possibility exists that, on its own display, information regarding the operation of a further cooking device can also be displayed. In this way, for example, it is possible to monitor the operation of two cooking devices from one cooking device.

The disclosure furthermore concerns a system which comprises at least two cooking devices according to the type named above, which are provided with means for transferring data between the control units of the two cooking devices. These means for transferring control signals can be achieved by signal lines, for example USB, Ethernet, RS232 or RS485 connections, or by a wireless connection, for example a Bluetooth connection.

Preferably the data are not processed between their production in the control unit of a cooking device and their receipt in the control unit of a further cooking device. In this embodiment thus no additional data processing equipment has to be connected between the cooking devices. Accordingly, in this embodiment, for example, no central server is necessary for controlling the operation of the cooking devices. Instead of that, the control units of the individual cooking devices have sufficient means for producing the relevant data, sending them, receiving them and converting them. In this way, cooking devices that operate essentially self- sufficiently, can, if needed, simply be connected to one another in order to be able to utilize the additional function of synchronization of the cooking operations.

### BRIEF DESCRIPTION OF THE DRAWINGS

Other advantages and characteristics of the cooking device according to the disclosure, of the system and of the method of the disclosure follow from the description which is given below, in which the disclosure is described with the aid of a preferred. embodiment, with reference to the attached drawing. The following is shown:

FIG. 1 is a schematic representation of a system of three cooking devices according to a further development of the disclosure.

### DETAILED DESCRIPTION OF A PREFERRED EMBODIMENT

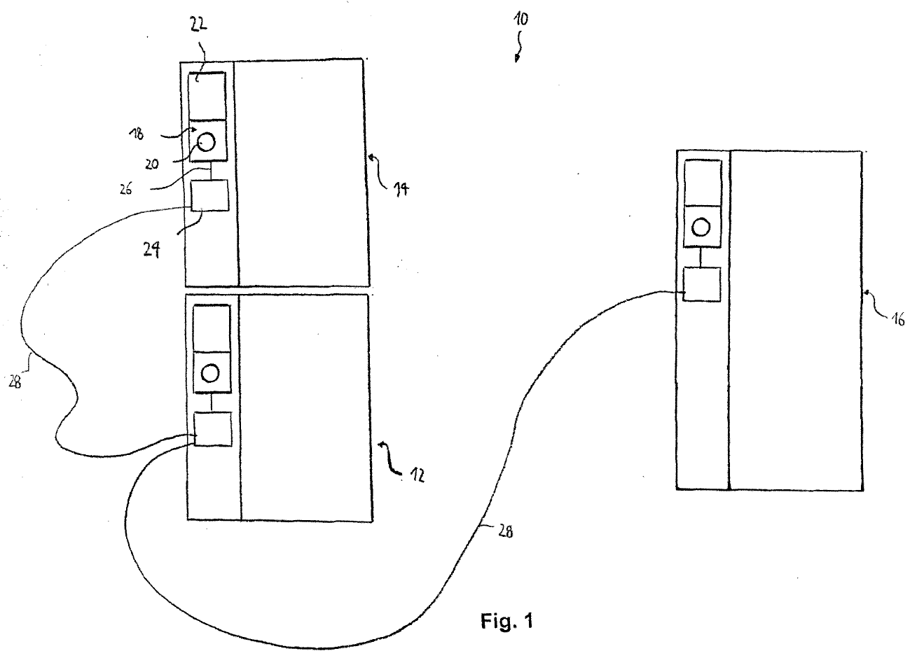

FIG. 1 shows a system 10 which comprises three cooking devices 12, 14 and 16. In the practical example shown, the cooking devices 12 and 14 which have the same construction are stacked on top of one another in order to save space. The cooking device 16 has fundamentally the same construction as cooking devices 12 and 14, but is somewhat larger.

Each of cooking devices 12, 14, 16 has an input device 18 with input means, of which the figure shows only a rotary switch 20 for selecting a cooking program, schematically. Furthermore, each of cooking devices 12, 14, 16 has a display 22 which can be, for example, an LCD display.

Finally, each of the cooking devices 12, 14, 16 has a control unit 24, which is suitable for controlling the cooking operation of the corresponding device in the known manner. Such a control unit 24 comprises, for example, one or several microprocessors and one or several memory devices in which programs are stored, with which the cooking operation can be controlled.

Each of the control units 24 is connected through a data line 26 to the input device 18 and the display 22 as an output device. Through this data line 26, commands are transferred from the input device 18, for example by the selection of a certain cooking program, to the control unit 24. Furthermore, information regarding the state of operation, for example temperature or moisture in the cooking chamber of the cooking device, can be transferred from the control unit 24 to the display 22 and displayed there.

The described combination of input device 18, display 22 and control unit 24 allows self-sufficient operation of each of the indicated cooking devices 12, 14, 16 in the conventional manner. In addition, however, the control units 24 of the cooking devices 12, 14, 16 are also suitable for producing data for the other cooking devices with which the operation of these other cooking devices can be influenced. Furthermore, the control units 24 of cooking devices 12, 14, 16 are designed to receive such data from the further cooking devices and to control their own operation in dependence on these received data. In order to exchange these additional data between the control units 24 of the cooking devices 12, 14, 16, in the practical example of FIG. 1, these devices are connected through data lines 29. In the practical example of FIG. 1, the control unit 24 of the cooking device 12 is connected to that of the cooking device 14 and of the cooking device 16. Additionally, the control units 24 of the cooking devices 14, 16 could be connected to one another, by means of which a broadcast would be produced. Instead of the data lines 28, wireless connections could also be provided between the control units 24.

In the preferred embodiment shown, the control units 24 of the different cooking devices 12, 14, 16 communicate in the type of a peer-to-peer network. In this joining of the control units 24, all control units 24 are fundamentally participating equally. The communication is done according to a broadcasting protocol. Hereby, all data packets which are sent by a control unit 24 are transferred to all other control units 24 of the system 10. These broadcasts are also called “broadcasting telegrams” in computer technology.

The transferred data concern such information or commands which are suitable for influencing the operation of the further cooking devices, so that the cooking devices 12, 14, 16 of the system 10 are synchronized with one another in a useful manner. The control unit 24 of each cooking device 12, 14, 16 takes from the broadcasting telegram those data which are relevant for the control of its own cooking device and controls the cooking device 12, 14, 16 correspondingly. Thus, no device that would communicate between the cooking devices has to be connected in-between.

The exchange of data between the control units 24 of the individual cooking devices 12, 14, 16 allows adjustment of the operation of these cooking devices 12, 14, 16 to one another or their synchronization. In order to illustrate the disclosure, three examples of the synchronization of the operation and the corresponding data are described below.

Locking function

In the practical example of FIG. 1, each of the cooking devices 12, 14, 16 has a frying function. Let us assume that a maximum frying output in the kitchen in which cooking devices. 12, 14, 16 are set up should, not be exceeded for reasons of safety. Let us assume that the frying output of each of the cooking devices 12, 14, 16 lies below this maximum permissible frying output, but the frying output of even two of the devices 12, 14, 16 already exceeds this maximum frying output.

When an operator chooses the frying function, for example in cooking device 12, in this embodiment the control unit 24 of the cooking device 12 produces a lock command and sends this to cooking device 14 as well as to cooking device 16. When during the validity period of this locking command, this or another operator selects a frying function on the remaining cooking devices 14, 16, this is denied due to the lock command. In this way it is ensured that frying can be done only on one cooking device at a time, so that a maximum permissible frying output in the system 10 can never be exceeded.

With the aid of this relatively simple measure, the need, for example, for a stationary fire extinguisher device can be eliminated, which otherwise would have to be provided for safety reasons when the frying output of the system 10 would exceed the maximum frying output. In this way, in a simple manner, additional construction costs for the kitchen in which the cooking devices 12, 14, 16 are installed can be avoided.

The locking command for the frying function is an example of the synchronization of the cooking devices 12, 14, 16, which consists in the fact that simultaneous frying in two of the three cooking devices 12, 14, 16, is avoided.

In an alternative embodiment, the maximum frying output is not limited by lock commands, but by the fact that the control units 24 of the cooking devices 12, 14, 16 merely inform the further cooking devices that they are frying with a certain frying output. Since in this embodiment this information in the broadcasting protocol is sent to all cooking devices 12, 14, 16, the control unit 24 of each of the cooking devices knows at which level of output frying is currently taking place. Furthermore, the difference between the maximum permissible flying output and the current total frying output can be determined in the system 10, and it can be decided if the remaining frying output is sufficient or not for its own frying operation.

This embodiment is especially flexible and can be extended easily because different devices with different frying outputs can be integrated into the system simply. The frying output is an example of a limited resource available for the entire system. Through the communication among the cooking devices 12, 14, 16, according to the disclosure, it is possible to divide such resources among the participating devices 12, 14, 16 without an operator having to actively concern himself with the matter

Identical cooking conditions in two devices

In order to explain this practical example, let us consider the case that a certain food is to be prepared in cooking device 14, whose holding capacity, however, is not sufficient for the desired quantity . In this case, the food can be divided between the cooking device 14 and the cooking device 12. Then, for example, the corresponding cooking program is entered in the accustomed manner to input device 18 of the cooking device 14. Furthermore, a command is entered into the input device 18 of the cooking device 14 (for example, a menu item is chosen), according to which the cooking process should proceed identically to that in the cooking device 12. Then, the control unit 24 of the cooking device 14 produces data during the cooking operation and sends it to the cooking device 12, in order to make sure that the cooking operation runs identically in the cooking device 12. For example, these control parameters could be the climate parameters of the cooking chamber of the cooking device 14, that is, for example, temperature, humidity and circulating air velocity. The control unit 24 of the cooking device 12 is then able to control the cooking process in the cooking device 12 correspondingly based on these signals.

From the point of view of the user, the consequence of this is effectively that the cooking devices 12 and 14 can be used together as one cooking device of twice the capacity. For the operator, this has the advantage that he has to operate only one of the cooking devices, in the present case, the cooking device 14. Without communication between the control units 24 of the cooking devices 12 and 14, the operator would have to manually adjust the cooking process in both devices 12, 14 and always make sure that they run identically so that the foods in both cooking devices 12, 14 are cooked uniformly.

Thus, in this practical example the synchronization of the cooking processes consists in the fact that the cooking device 12 produces an exact copy of the cooking process of the cooking device 14.

Temporal synchronization of cooking processes

Another practical example concerns the case in which different foods are to be cooked in different cooking devices 12, 14, 16, but at the same time and possibly they even must be finished at a certain preprogrammed time in order that they can be served together. In an embodiment of the disclosure, one of the cooking devices 12, 14, 16 sends data or control signals to the further cooking devices, which represent a time or time period at which or within which their cooking process should be completed in all the participating cooking devices 12, 14, 16. In one embodiment the operator decides which of the cooking devices 12, 14, 16 will send these control signals to the other participating cooking devices. In an alternative embodiment, automatically that cooking device is chosen for which it is predicted that it will require the longest cooking process and this then sends the control signals to the other participating cooking devices.

However, each of the participating cooking devices can send messages to the further cooking devices at any time. If, for example, a delay occurs in one of the cooking devices, for example, because the cooking chamber was not closed correctly or because a brief deficiency of water occurred, then this cooking device sends a corresponding broadcasting telegram to the participating cooking devices. The control units 24 of the other participating cooking devices recognize that there is a delay and adjust their cooking operation so that the cooking process will end in all participating cooking devices simultaneously after the elimination of the error or after the end of the disturbance.

In this embodiment, the operator introduces the foods into the participating cooking devices, and at each cooking device selects the desired cooking process in the usual manner with the input device 18. The temporal synchronization of these cooking processes will then, however, be synchronized by the control units 24 which, in the manner described above, exchange time information and possibly other information, for example, error messages. With the aid of the time information, the control unit 24 of each cooking device can control the cooking process in the corresponding cooking device in such a way that all the cooking processes are completed simultaneously.

In this practical example the synchronization of the cooking processes thus consists in the fact that the end of the cooking processes in time in the different cooking devices 12, 14, 16 agree even if a temporary disturbance should occur in one of the devices. For the operator this has the advantage that he is merely required to select the optimum cooking programs in the participating cooking devices 12, 14, 16, but does not have to concern himself with the temporal synchronization of the cooking processes.

Other properties of the system

In addition, the connection of the control units 24 with signal lines 28 has the advantage that the cooking devices connected to one another can use their resources mutually. For example, when a large number of foods are introduced into cooking device 14, the conditions. of which have to be controlled and monitored individually (so-called level control), it may happen that the display 22 of the cooking device 14 is not sufficient to represent the information of interest for all of the introduced foods. However, if at the same time the cooking device 12 is not in operation, the displays 22 of the cooking device 12 can be used to present the remaining information. In this case, thus, the display 22 of the cooking device 12 is used to display operating information regarding the cooking device 14.

As an alternative example let us consider the case in which cooking devices 14 and 16 are in operation, but, at the present time the cooking device 12 is not being utilized. In this case it is possible to send the output information of the cooking device 16 to the display 22 of the cooking device 12. This is advantageous for the operator since the cooking devices 12 and 14 are close to one another, namely they are stacked on top of one another, and the operator can monitor the displays 22 of the cooking devices 12, 14 simultaneously. By contrast, in a completely separate operation, the operator would have to go back and forth continuously between the cooking devices 16 and 14 in order to monitor the cooking operation.

In the described practical example of FIG. 1, the synchronization of the different cooking devices 12, 14, 16 is achieved by communication of the control units 24 of the cooking devices 12, 14, 16, which are also responsible for the local control of the corresponding device. Thus, in this practical example, for the synchronization of the cooking devices, the functionality of the control units 24 is thus extended in comparison to the conventional cooking devices. However, no central control unit, for example a central server, is necessary. This has advantages with regard to installation as well as with regard to operation. The operators can operate the cooking devices 12, 14, 16 in the usual manner through the input devices 18, as if the cooking devices were not connected to one another. However, moreover, there are additional functions which can be selected through the same input device 18 which permit the synchronization of the cooking devices. However, the operators do not have to be familiar with an additional input device, for example, a central computer, in order to control the cooking devices, which would have made the operation more complicated and reduce acceptance.

The characteristics described above may be of importance in any arbitrary combination.

1-28. (canceled)

29. Cooking device with a control unit, whereby the control unit comprises a memory, a processor, and one or more programs stored on the memory, the one or more programs being executable by the processor for:
producing data suitable to synchronize the operation of a further cooking device with the operation of its own cooking device and to transfer the data to the further cooking device when it is communicatively connected with this further cooking device, and
receiving data from a further cooking device and to control the operation of its own cooking device in dependence on the data in such a way that the operation of its own cooking device is synchronized with the operation of the further cooking device,
wherein the program is further executable by the processor of the control unit for sending the data in a broadcast to control units of all cooking devices with which the control unit is communicatively connected, receiving a broadcast that was sent by the control unit of a further cooking device, and extracting data from the broadcast, the extracted data being relevant for the operation of the control unit's own cooking device and for controlling the corresponding cooking device in accordance with the extracted data.

30. Cooking device according to claim 29, in which the one or more programs are further executable by processor of the control unit for transferring data to the further cooking device without the interconnection of a further data processing unit.

31. Cooking device according to claim 29, in which the control unit for producing data for a further cooking device is identical with the control unit used for controlling the cooking operation of its own cooking device.

32. Cooking device according to claim 29, in which the data represent at least one of the following information or commands:
time information, which displays at which time or within which time period a cooking process should be finalized,
information concerning the climate parameters in the cooking chamber,
cooking commands which initiate certain cooking functions in the data- receiving cooking device,
locking commands which lock certain functions in the data-receiving cooking device,
information concerning resources which are used by a cooking device, and error messages.

33. Cooking device according to claim 32, wherein the data representing information concerning the climate parameters in the cooking chamber represent at least one of a temperature, a moisture, and a circulating air velocity in the cooking chamber.

34. Cooking device according to claim 32, whereby one function that can be locked by a locking command is a frying function, or one resource that is utilized by a cooking device is represented by a frying output.

35. Cooking device according to claim 29, in which the data are suitable to influence the operation in two cooking devices in such a way that the cooking conditions in the two cooking devices correspond to one another, and/or in which the control unit is suitable for controlling the corresponding cooking device after receipt of time information from a further cooking device in such a way that the cooking process in the corresponding cooking device finished in accordance with the time information.

36. Cooking device according to claim 29, further comprising an input device for entering cooking commands for the corresponding cooking device and for entering information that concerns the synchronization of the operation of the corresponding cooking device with at least one further cooking device, and/or with a display for displaying information concerning the operation of the input device's own cooking device, whereby the control unit of the input device's own cooking device is suitable for displaying information concerning the operation of a further cooking device on the display device.

37. System comprising at least two cooking devices according to claim 29, comprising means for transferring data between control units of the at least two cooking devices.

38. System according to claim 37, in which the means for transferring the control signals comprises signal lines, and/or in which the data are not processed between their production in the control unit of a cooking device and their receipt in the control unit of a further cooking device.

39. System according to claim 38, in which the means for transferring the control signals comprises a USB connection, an Ethernet connection, an RS232 connection, an RS485 connection, or a wireless connection.

40. System according to claim 39, wherein the wireless connection comprises a Bluetooth connection, a WLAN connection, a WiFi connection, a UMTS connection, a GPRS connection, an EDGE connection, or a modem connection.

41. System according to claim 37, in which the control units of the at least two cooking devices are arranged for the communication through a peer-to- peer connection.

42. System according to claim 41, wherein the control units of the at least two cooking devices are arranged for communication via a broadcasting protocol according to which each control unit sends the produced data to the control units of all further cooking devices of the system and all data sent in the system are received by the control unit of each cooking device.

43. Method for controlling at least two cooking devices, each with a control unit according to claim 29, wherein the control unit sends the data in a broadcast to the control units of all cooking devices with which it is connected, the control unit of each further cooking device receives the broadcast and extracts from the broadcast the data that are relevant for the operation of its own cooking device and controls the corresponding cooking device according to the extracted data.

44. Method according to claim 43, in which the data are produced in such a way that at least approximately identical cooking conditions are created simultaneously in at least two cooking devices.

45. Method according to claim 43, in which the data are produced in such a way that cooking processes in different cooking devices are finished at least approximately simultaneously.

46. Method according to claim 45, wherein the cooking unit of one of the cooking devices predetermines a time or time period at which or within which the cooking process in the corresponding cooking device ends, data which represent this time or time period are produced in the control device of this cooking device, the data are transferred to the control unit of at least one further cooking device and the cooking process in this at least one further cooking device is controlled in dependence on these data in such a way that it also ends at this time or within this time period.

47. Method according to claim 43, in which information which concerns the synchronization of the operation of a present cooking device with that of at least one further cooking device is entered into an input device, which is the same as an input device that receives operating commands concerning the operation of the present cooking device.

48. Method according to claim 43, in which a display device of an unused cooking device is used to display operating information of a further cooking device.
